(19) United States
(12) Patent Application Publication
(10) Pub. No.: US 2006/0231108 A1
(43) Pub. Date: Oct. 19, 2006

(54) Method and apparatus for managing multi-patient contexts on a picture archiving and communication system

(75) Inventors: Benjamin D. Novatzky, Oak Park, IL (US); Himanshu Mehta, Chicago, IL (US); Kengo Baba, Evanston, IL (US); James Jay, Lake Villa, IL (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/108,228

(22) Filed: Apr. 18, 2005

(51) Int. Cl. A61B 5/00

(52) U.S. Cl. 128/898; 600/300

(57) ABSTRACT

A system and method to increase the efficiency and effectiveness of a radiologist. A context manager for managing the display of multi-patient contexts. In an embodiment computer software operates as a context manager and correlates the context of at least one information container with at least one image container. A graphical container displaying information about a patient, as opposed to an image, may be an information container. A graphical container displaying images of a patient, as opposed to information, may be an image container. In order to minimize occurrences of a user viewing information about a first patient and images of a second patient, and mistakenly believing the information and images belong to the same patient, a visual indicator may be displayed over images that are out-of-context with the information displayed. The visual indicator may be a watermark to alert a user of a difference in context.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to a system and method for improved medical imaging. Particularly, the present invention relates to a more efficient system and method for managing the display of medical images and data.

Medical diagnostic imaging systems encompass a variety of imaging modalities, such as x-ray systems, computerized tomography (CT) systems, ultrasound systems, electron beam tomography (EBT) systems, magnetic resonance (MR) systems, and the like. Medical diagnostic imaging systems generate images of an object, such as a patient, for example, through exposure to an energy source, such as x-rays passing through a patient, for example. The generated images may be used for many purposes. For instance, internal defects in an object may be detected. Additionally, changes in internal structure or alignment may be determined. Fluid flow within an object may also be represented. Furthermore, the image may show the presence or absence of objects in an object. The information gained from medical diagnostic imaging has applications in many fields, including medicine and manufacturing.

An example of a medical diagnostic imaging system is Picture Archival Communication Systems (PACS). PACS is a term for equipment and software that permits images, such as x-rays, ultrasound, CT, MRI, EBT, MR, or nuclear medicine for example, to be electronically acquired, stored and transmitted for viewing. In addition to images, information about a patient and/or study may also be acquired, stored, and transmitted for viewing.

The information and images may be organized according to the context of the information and images. For example, the context of a patient may be images of a patient, the patient's name, date-of-birth, medical history, and home address. Moreover, the context of a study may be information and images related to a particular study of a particular patient. For example, images of a patient from a particular study, procedure type, study date/time, and study status are examples of a study context.

A PACS system typically provides a user interface capable of displaying images of a patient and/or study simultaneously with the associated information about the patient and/or study. The PACS user interface also typically provides the ability to display multiple studies simultaneously. Multiple studies of a single patient or studies of different patients may be displayed. Typically, when displaying multiple studies, multiple images are displayed on a display unit.

Displaying images of different patients simultaneously, however, may present dangerous scenarios. A radiologist, clinician, technologist, or other PACS user may potentially be viewing images belonging to a first patient, viewing the patient history from a second patient, and viewing a record of a third patient, all at the same time. Accordingly the user may be reviewing three distinct patient and/or study contexts without even realizing it. Such scenarios may result in a user attributing information and/or images for a first patient to a second patient. For example, a user having information and images open for Patients A and B may attribute images of Patient A to Patient B. Such user errors present dangerous scenarios of misinterpretation and misdiagnosis.

Therefore, a need exists for a system and method for managing multi-patient contexts. Such a system and method may promote synchronization of images and information displayed for the same patient and/or study context. Such a system and method may alert a user to images or data displayed out-of-context, helping to minimize user errors.

### SUMMARY OF THE INVENTION

Certain embodiments of the present invention provide a system and method for displaying medical images. In an embodiment a computer unit manipulates data using computer software to operate as a context manager for correlating the context of at least one information container with at least one image container. The system utilizes at least one display unit for displaying at least one information container and at least one image container. A user may select a context driver.

If an information container is the context driver, the other information containers synchronize study context with the study context of the context driver. The image containers having patient contexts different from the patient context of the context driver display a visual indicator over the medial images. The visual indicator may be a watermark.

If an image container is a context driver, the other image containers having a patient context different from the patient context of the context driver display a visual indicator over said medical images. The visual indicator may be a watermark. The information containers may synchronize with the study context of the context driver.

The information containers may contain an image icon for indicating the studies displayed in the image containers. Moreover, the image containers may have a quick show feature for displaying an information container from an image container. The quick show feature may permit the display of a report container and a patient work list container. The quick show container may be displayed by hovering a computer mouse over a button. The quick show containers may also be pinned to lock the display of the quick show containers.

Certain embodiments of the present invention provide a method for displaying medical images. First, a container is selected as a context driver. Next, a context is selected. Next, the context of at least one information container synchronizes according to the context of the context driver. Finally, a visual indicator is displayed over an image in an image container wherein the patient context of the image container differs from the patient context of the context driver. The visual indicator may be a watermark. The information containers may contain an image icon for indicating the studies displayed in the image containers. Moreover, the image containers may have a quick show feature for displaying an information container from an image container. The quick show feature may permit the display of a report container and a patient work list container. The quick show container may be displayed by hovering a computer mouse over a button. The quick show containers may also be pinned to lock the display of the quick show containers.

Certain embodiments also include a computer—readable storage medium including a set of instructions for a computer. The set of instructions include a first selection routine for selecting a container as the context driver and a second selection routine for selecting the context. The set of instructions also include a synchronization routine for synchronizing the study context of at least one information container according to the study context of the context driver. The set of instructions also include, a first display routine for displaying a visual indicator over an image displayed in an image container wherein the patient context of the image container differs from the patient context of the context driver. The set of instructions may also include a second display routine for displaying an image icon indicating the studies displayed in the image containers. The set of instructions may also include a third display routine for displaying a quick show window for displaying an information container from an image container.

### BRIEF DESCRIPTION OF THE DRAWINGS

FIG. 1 illustrates an example of a medical imaging system which may be used in accordance with an embodiment of the present invention.

FIG. 2 illustrates a graphical application of a system displaying a plurality of user interface containers in accordance with an embodiment of the present invention.

FIG. 3 illustrates a graphical application of a system displaying a plurality of user interface containers in accordance with an embodiment of the present invention.

FIG. 4 illustrates a graphical application of a system displaying a plurality of user interface containers in accordance with an embodiment of the present invention.

FIG. 5 illustrates a method in accordance with an embodiment of the present invention.

FIG. 6 illustrates a graphical application of a system displaying a user interface container showing an embodiment of the quick view feature.

FIG. 7 illustrates a graphical application of a system displaying a user interface container showing an embodiment of the quick view feature.

### DETAILED DESCRIPTION OF THE INVENTION

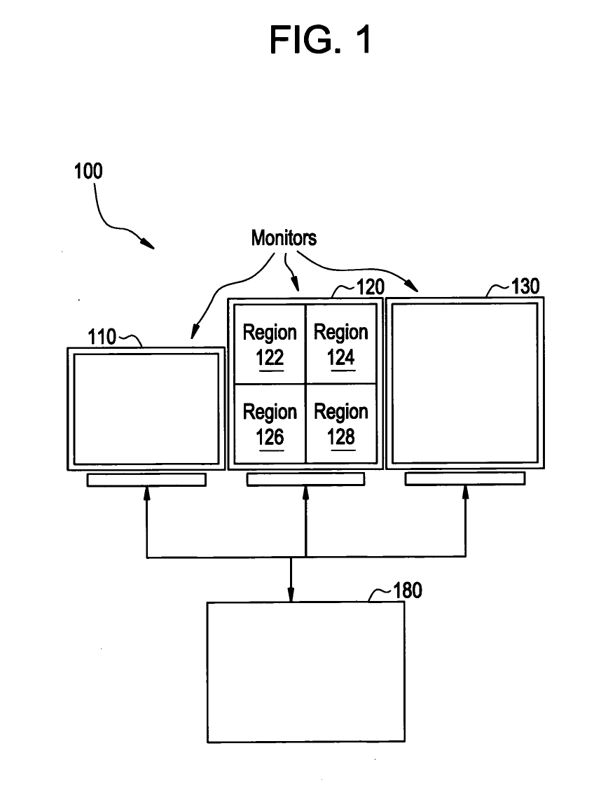

FIG. 1 illustrates a system 100. The system 100 an is example of a medical imaging system that may be used in accordance with an embodiment of the present invention. As an example, the system 100 shows three display units, unit 110, unit 120, and unit 130. The display units 110-130 may be computer monitors or other display devices. Units 110 and 130 may include a single display region, or a plurality of display regions. Unit 120 includes four display regions, 122, 124, 126, and 128, respectively. Although three display units are shown in the system 100, the invention may be realized with any number of display units, including a single display unit.

Also included in the system 100 is a computer unit 180. The computer unit 180 may be any equipment or software that permits electronic medical images and data, such as x-rays, ultrasound, CT, MRI, EBT, MR, or nuclear medicine for example, to be electronically acquired, stored, or transmitted for viewing and operation. The computer unit 180 may be connected to other devices as part of an electronic network. In an embodiment, the computer unit 180 may be, or may be part of, a picture archival communication system (PACS). The computer unit 180 may represent equipment that may be generally associated with PACS, including input controls, and communication, processing, and storage equipment, or other associated equipment. The system 100 may be a PACS with display units 110, 120, and 130. The computer unit 180 and display units 110, 120, and 130 may also be separate units. In such a case, the display units 110, 120, and 130 may be in electrical communication with the computer unit 180. The components of the system 100 may be single units, separate units, may be integrated in various forms, and may be implemented in hardware and/or in software.

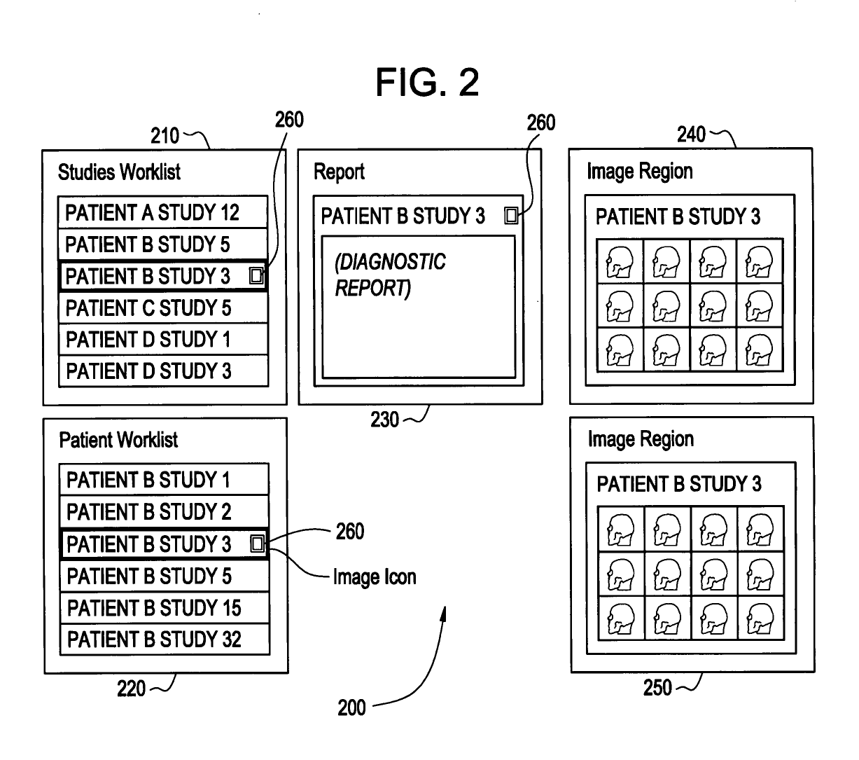

FIG. 1 is just an example, and a greater number or lesser number of display units may be used in accordance with the present invention. FIGS. 2-7 illustrate an example of computer software that may operate as part of, or some variation of, the system 100. Specifically, FIGS. 2-7 illustrate computer software operating as a context manager for correlating the context of available information with available images. Accordingly, as FIGS. 1-7 are an example, any hanging protocol may be used and any combination of images, information, and data may be displayed in accordance with the present invention.

FIG. 2 illustrates a system 200 in accordance with an embodiment of the present invention. The system 200 includes a plurality of user interface containers 210-250 that may be displayed on display units 110-130 in the system 100. For example, containers illustrating information, as opposed to images, may be the studies work list container 210, the patient work list container, 220 and the report container 230. Containers illustrating images may be containers 240 and 250. As an example, containers 210-230 may be displayed in some combination on regions 122-128 of display unit 120. Image containers 240 and 250 may be displayed on display units 110 and 130. However, the system as described is only an example. The information containers 210-230 and image containers 240-250 may have any hanging protocol, including displaying the containers 210-250 on a single display unit.

The studies work list container 210 may be an information container that displays a list of studies, potentially belonging to different patients. The listed studies may be recently viewed studies, related studies, comparison studies, all studies at an institution, or some other combination of studies. The current study selected in the studies work list container 210 may define the patient context and study context of the studies work list container 210. For example, the study PATIENT B STUDY 3 is highlighted in the studies work list container 210. The patient context of the studies work list container 210 is PATIENT B. The study context of the studies work list container 210 is STUDY 3. As shown in FIG. 2, the studies work list container 210 provides a user with the ability to select a context. Accordingly, the studies work list container 210 may be a context driver.

The patient work list container 220 may be an information container that displays a list of available studies belonging to a single patient. The current study selected in the patient work list container 220 may define the patient context and study context of the patient work list container 220. For example, the study PATIENT B STUDY 3 is highlighted in the patient work list container 220. The patient context of the patient work list container 220 is PATIENT B. The study context of the patient work list container 220 is STUDY 3 of PATIENT B. As shown in FIG. 2, the patient work list container 220 provides a user with the ability to select a context. Accordingly, the patient work list container 220 may be a context driver.

The report container 230 may be an information container that displays a diagnostic report belonging to a study. The context of the report container 230 may consist of the patient and study to which the report belongs. In an embodiment, the context of the report container 230 is driven by the patient context and study context of the studies work list container 210 or patient work list container 220. For example, the patient context of the studies work list container 210 is PATIENT B. The study context of the studies work list container 210 is STUDY 3 of PATIENT B. In an embodiment, the report container 230 may synchronize with both the patient and study context of the studies work list container 210. In another embodiment, the report container 230 synchronizes with the patient context of the studies work list container 210. In either embodiment, the report container does not offer the ability to select a context. The report container 230 receives context from the studies work list container 210 or patient work list container 220. Accordingly, the report container may not be a context driver.

The image containers 240 and 250 are image containers that may display images from a study of a patient. One container may generally display images from a particular study of a single patient. However, a study may contain more images than may be displayed in a typical container and the images of a study may be displayed in more than one container. The context of the image containers 240 and 250 may be based on the patient and/or study whose images are displayed. For example, the patient context of the image container 240 is PATEINT B and the study context is STUDY 3 of PATIENT B. The patient context of the image container 250 is PATIENT B and the study context is STUDY 3 of PATIENT B. A user may select context of the image containers 240 and 250 by selecting an image container as active, for example clicking on the container with a computer mouse. Accordingly, the image containers 240 and 250 may be context drivers.

Also shown in FIG. 2 is the image icon 260. The image icon 260 may be an icon displayed as part of the studies work list container 210 and/or the patient work list container 220 and/or the report container 230 as an indicator of open image containers. For example, the image icon 260 is displayed in FIG. 2 as part of the patient work list container 220 at PATIENT B STUDY 3, studies work list container 210 at PATIENT B STUDY 3, and the report container 230 next to the report of PATIENT B STUDY 3. The image icon 260 indicates that images for PATIENT B STUDY 3 are displayed in an image container. In the example shown in FIG. 2, images for PATIENT B STUDY 3 are displayed in image container 240 and image container 250.

In the example as shown in FIG. 2, the containers 210-250 have the same patient context and study context. For example, the patient context of the information containers 210-230 is PATIENT B. The patient context of the image containers 240 and 250 are also of PATIENT B. The study context of the information containers 210-230 is STUDY 3 of PATIENT B. The study context of the image containers 240 and 250 is also STUDY 3 of PATIENT B. In an embodiment, a user may choose to manage both the patient context of the containers displayed and/or the study context of the containers displayed. If a user manages the container context based on a particular patient, in other words on a patient context, then the context of the containers is managed so both information and images correlate to the particular patient. If a user manages the container context based on a particular study of a particular patient, in other words a study context, then the context of the containers is managed so both information and images correlate to a particular patient and study. In either case, image containers display out-of-context visual indicators to indicate that the images displayed within the image container may be out-of-context with the information containers.

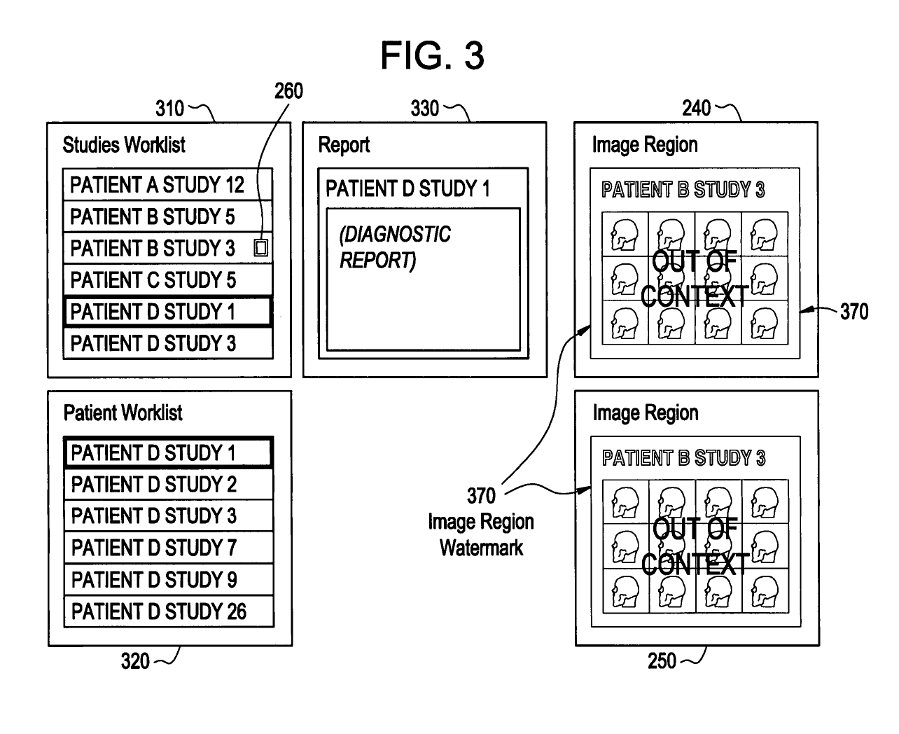

FIG. 3 illustrates a system 300 in accordance with an embodiment of the present invention. The system 300 illustrates the same containers as system 200, however a user has changed the context. The context of the system 200 is PATIENT B STUDY 3. The context of the system 300 is PATIENT D STUDY 1. Containers illustrating the new context have been marked 310, 320, and 330, respectively. The image containers 240 and 250 have not adopted the context of PATIENT D STUDY 1, so the images displayed as part of the image containers 240 and 250 have a visual indicator indicating the images are out-of-context. In the embodiment shown in FIG. 3, the visual indicator is an image region watermark 370. The visual indicator may be any sort of indicator, including a dimming pane displayed over the images, text displayed over the images, or both a dimming pane and text displayed over the images.

Also note, the image icon 260 is still displayed in studies work list container 310 next to PATIENT B STUDY 3, even though the context of the information containers 310-330 has been changed to PATIENT D STUDY 1. The image icon 260 indicates the images displayed in the image containers. The image containers 240 and 250 are still displayed even though the images are now out-of-context with the information containers.

In an embodiment, a new context may be selected using a context driver. The context driver may be a container which allows a user to select a new context. For example, the studies work list containers 210/310 and patient work list containers 220/320 allow a user to select a new context from a list. The image containers 240 and 250 allow a user to select the context of the images displayed in the image container. For example, the image containers 240 and 250 permit selection of context PATIENT B STUDY 3.

The context of a system may be managed based on whether an information container or a image container is the context driver. In the change of context from system 200 to system 300, an information container was the context driver. When an information container is the context driver, the other information containers synchronize with the study context of the context driver. In other words, the other information containers not only synchronize to show the same patient, but also the same study as the context driver. The image containers whose patient contexts differ from the context of the patient context of the driver context, display a visual indicator, such as a watermark, over the images indicating the images are out-of-context.

Note that in an embodiment, the image containers display the watermark when the patient context differs from the patient context of the context driver. For example, in FIG. 2 if the user selects a new study from the patient work list container 220 for PATIENT B, the other information containers may synchronize to represent the change of study context. However, the patient context, PATIENT B, has remained the same, so the image containers may not display a watermark. In other words, the image containers may display a watermark if the patient context of the image containers is different than the patient context of the information containers.

Accordingly, a user may simultaneously view and compare the images of different studies belonging to the same patient without obstructive watermarks. However, watermarks may be displayed to minimize the potential for a user to inadvertently associate the images of one patient with the report/records of another patient.

If an image container is selected as the context driver, the information containers having contexts different from the context of the context driver synchronizes with the context of the context driver. For example, if the user activates the image container 240 in FIG. 3 by clicking on the image container 240 with a mouse, for example, the context of the studies work list container 310, patient work list container 320, and report container 330 would synchronize with the context of the image container 240 to PATIENT B STUDY 3. The other image containers having patient contexts different from the patient context of the context driver may display a visually apparent watermark over the images. For example, in FIG. 3, if a user were to click on image container 240 as the context driver, because image container 250 has the same patient context, PATIENT B, as the context driver, the watermark would be removed and the image displayed. Again note the other image containers may display the watermark when the patient context of the image container differs from the patient context of the context driver.

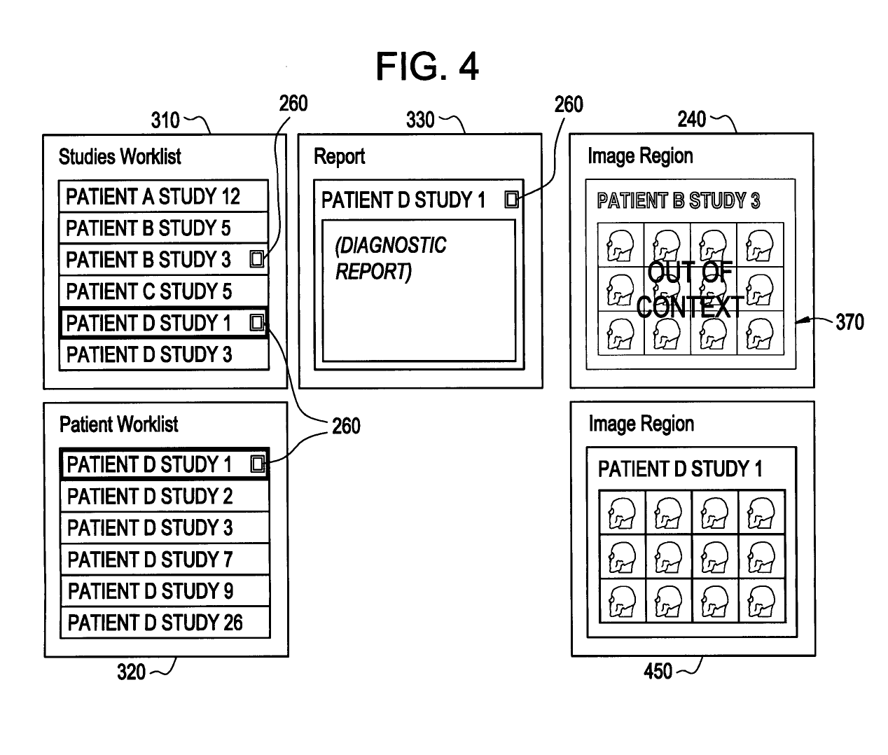

FIG. 4 illustrates a system 400 in accordance with an embodiment of the present invention. The system 400 illustrates a system similar to system 300 as the context is still PATIENT D STUDY 1. However, a user has selected to open the image container 450 for PATIENT D STUDY 1 and leave the image container 240 of PATIENT B STUDY 3 open. Accordingly, the image container 240 has patient context of PATENT B and image container 450 has patient context of PATIENT D. The patient context of image container 450 is the same as the patient context of the information containers 310-330, and therefore is displayed without a watermark. The patient context of image container 240 is different from the information regions 310-330, and therefore is displayed with a watermark indicating the images are out-of-context.

Also note that the image icon 260 now appears in front of PATIENT D STUDY 1 in both the studies work list 310 and patient work list 320 as well as in front of PATIENT B STUDY 3. As mentioned above, the display of the image icon 260 is displayed next to contexts open in image containers. Even though it is watermarked, the context of PATIENT B STUDY 3 is open in image container 240. The context of PATIENT D STUDY 1 is open in image container 450. Accordingly, the image icons 260 are displayed next to the appropriate contexts.

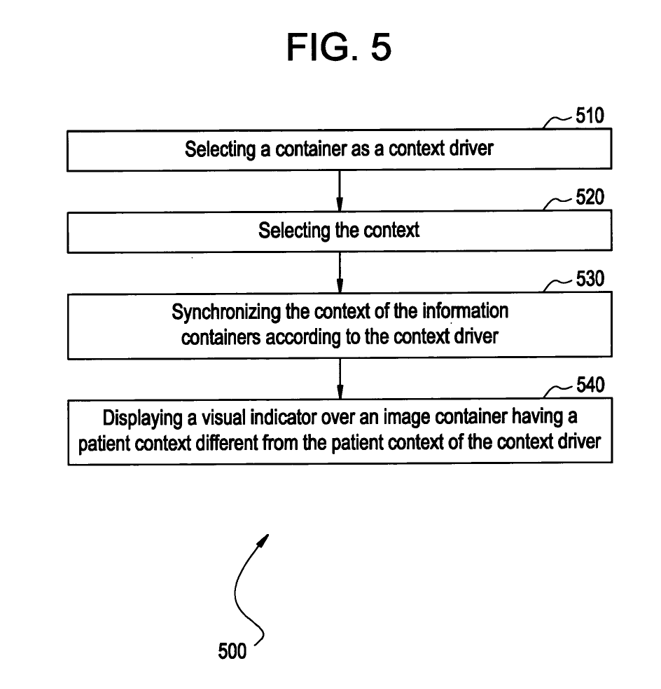

FIG. 5 illustrates a method 500 for managing multi-context displays. At step 510 a user selects a container as the context driver container. As mentioned above, the context driver may be a container that allows a user to select a new context. The context driver may be an information container or an image container. At step 520, the user selects a context. For example, the context may be selected from a list, as in the case of the information containers. The context may also be selected by selecting an image container, with a mouse for example, as in the case of the image containers. At step 530, the context of the information containers may be synchronized according to the context of the context driver. In an embodiment, the information containers synchronize with the study context of the context driver. Accordingly, the information containers may have the same patient context and study context as the context driver. Finally, at step 540, the image containers having a patient context different from the patient context of the context driver may display a visual indicator to indicate the images displayed within the container are out of context with the patient context of the information containers. In other words, the images displayed within the containers are images of a different patient than the patient of the current context.

The visual indicator discussed at step 540 may be a watermark. The watermark may be a dimmed pane to obscure the view of the images within the image container. In an embodiment, the dimmed pane may also include an image or text to indicate to a user that the images displayed are out-of-context with the information containers. Additionally, the method 500 may also include displaying an image icon within the information containers to indicate the display of a study in the image containers. The method 500 may also include displaying a quick show window, as is further discussed below.

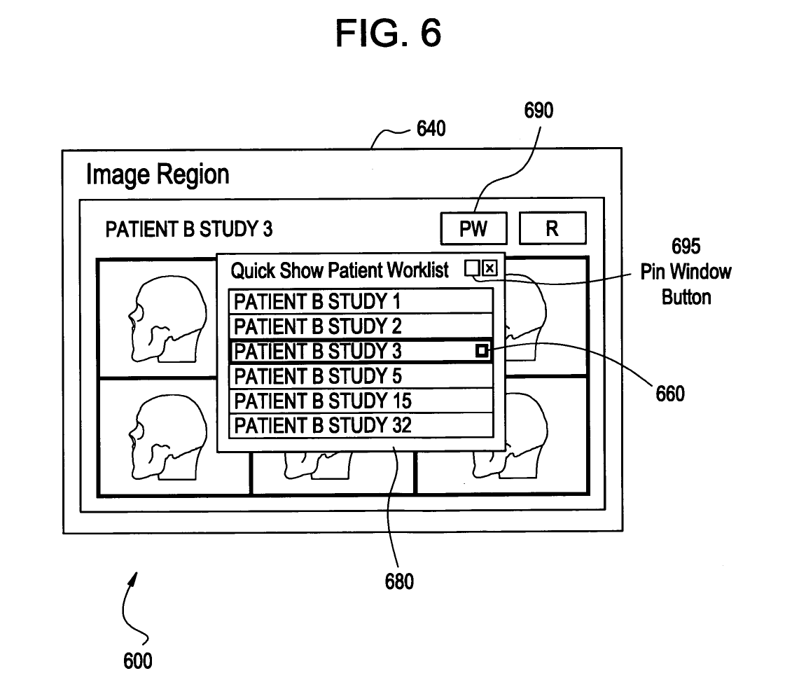

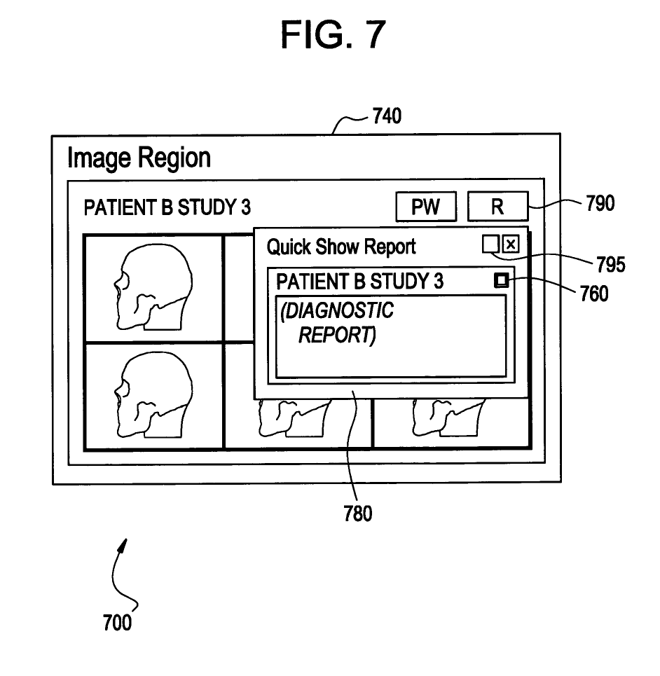

FIGS. 6 and 7 illustrate systems 600 and 700 in accordance with an embodiment of the present invention. The systems 600 and 700 may be employed similarly to systems 100-400 and method 500 previously discussed. Systems 600 and 700, however, add an additional feature to the image containers discussed in systems 100-400 and method 500. In an embodiment, the additional feature is an quick show window.

The quick show window showing in system 600 is window 680 and the quick show window in system 700 is window 780. In the embodiment shown, the window 680 may be a quick show patient work list 680. The quick show patient work list 680 may be the same patient work list as patient work list 220/320 previously discussed. The window 780 may be a quick show report 780. The quick show report 780 may be the same report as report 230/330 previously discussed.

In an embodiment, the quick show feature permits the display of an information container within or around an image container. The quick show windows 680 and 780 are displayed within the image containers 640 and 740 respectively. Also, although not shown, a study work list similar to the study work list 210/310 may be displayed as a quick show study work list.

In the system 600, a user may display the quick show patient work list 680 by selecting the quick show patient work list button 690. The user may generally hover the computer mouse over the quick show patient work list button 690 to display the quick show patient work list 680. In an embodiment, as long as the mouse is either over the quick show patient work list button 690 or over the quick show patient work list 680, the quick show patient work list 680 may be displayed. A user may also pin the quick show patient work list 680 open using the pin window button 695. When a window is pinned open, the window may remain open until the window is closed or the context has been changed.

The quick show feature illustrated in the system 600 provides a user the ability to change study context from an image container to a study context other than the study context of the image. For example, in the embodiment of system 600, a user may open the quick show patient work list 680 to select a new study context from the list. The selection of a new study context may cause the information containers to synchronize with the new study context. Note, however, that as the list of studies in the quick show patient work list 680 are studies of Patient B, the selection of a new study does not change the patient context. Accordingly, the selection of a new study from the list available in quick show patient work list 680 may not cause a visual indicator to be displayed over the image region of the image container 640. If the image container contained a quick show study work list, a change in patient context may cause a visual indicator to be displayed over the image region of the image container.

The quick show feature illustrated in the system 700 provides a user the ablity to access information about a patient and a study from the image containers in which the image are displayed. For example, in the embodiment illustrated in the system 700, a quick show report 780 may be displayed. The quick show report 780 may be displayed and pinned in a similar manner as the quick show patient work list 680. The quick show report 780 may be displayed and pinned with buttons 790 and 795, respectively. In an embodiment, a user may not change context through the quick show report 780. The quick show report 780 reports information about the context. In other words, the quick show report 780 reports information about the patient and study.

It should be noted that in systems 600 and 700, the display of the quick show window does not change the patient context. Moreover, the quick show windows may also be used to view information from a different context without actually changing the current context. Thus, a user may view the report of a study whose image is out-of-context with the current context. In other words, the user may view the report of a study whose images are watermarked. As the quick show window may not be displayed when the user moves the computer mouse away from the out-of-context image container, the potential for a user to associate the quick show report with another image container, which may belong to another patient, is minimized.

The system and method described above may be carried out as part of a computer—readable storage medium including a set of instructions for a computer. The set of instructions includes a selection routine for selecting a container as the context driver. As mentioned above, the context driver may be a container that allows a user to select a new context. The context driver may be an information container or an image container. The computer instructions also include a selection routine for selecting the context. For example, the context may be selected from a list, as in the case of the information containers. The context may also be selected by selecting an image container, with a mouse for example, as in the case of the image containers. The computer instructions also include a synchronization routine for synchronizing the context of the information containers according to the context driver. In an embodiment, the information containers synchronize with the study context of the context driver. Accordingly, the information containers may have the same patient context and study context as the context driver. The computer instructions also include a display routine for displaying a visual indicator over an image region of an image container having a patient context different from the patient context of the context driver. In an embodiment, the visual indicator may be a watermark.

Additionally, the set of instructions may also include a second display routine for displaying an image icon within the information containers to indicate the display of a study in the image containers. The set of instructions may also include a third display routine for displaying a quick show window.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A system for displaying medical images, said system comprising:
a computer unit for manipulating data, said computer unit having computer software operating as a context manager for correlating the context of at least one information container with at least one image container; and
at least one display unit for displaying at least one information container and at least one image container.

2. The system of claim 1, wherein said information container is a context driver, other information containers synchronize study context with the study context of said context driver.

3. The system of claim 2, wherein said image containers having a patient context different from the patient context of the context driver display a visual indicator over said medical images.

4. The system of claim 3, wherein said visual indicator is a watermark.

5. The system of claim 1, wherein said image container is a context driver, other image containers having patient a context different from the patient context of the context driver display a visual indicator over said medical images.

6. The system of claim 5, wherein information containers synchronize study context with the study context of said context driver.

7. The system of claim 5, wherein said visual indicator is a watermark.

8. The system of claim 1, wherein said information container contains an image icon indicating studies displayed in said image containers.

9. The system of claim 1, wherein said image container has a quick show feature for displaying an information container from an image container.

10. The system of claim 9, wherein said quick show feature permits display of a report container and a patient work list container.

11. The system of claim 9, wherein said quick show containers may be displayed by hovering a computer mouse over a button.

12. The system of claim 9, wherein said quick show information containers may be pinned.

13. A method for displaying medical images, said method comprising:
selecting a container as a context driver;
selecting a context;
synchronizing the study context of at least one information container according to the study context of the context driver; and
displaying a visual indicator over an image displayed in an image container wherein the patient context of said image container differs from the patient context of said context driver.

14. The method of claim 13, wherein said visual indicator is a watermark.

15. The method of claim 13, further comprising displaying an image icon indicating studies displayed in said image containers.

16. The method of claim 13, further comprising displaying a quick show window for displaying an information container from an image container.

17. The method of claim 16, wherein said quick show window permits display of a report container and a patient work list container.

18. A computer—readable storage medium including a set of instructions for a computer, the set of instructions comprising:
a first selection routine for selecting a container as the context driver;
a second selection routine for selecting the context;
a synchronization routine for synchronizing the study context of at least one information container according to the study context of the context driver; and
a first display routine for displaying a visual indicator over an image displayed in an image container wherein said patient context of said image container differs from the patient context of said context driver.

19. The set of instructions of claim 18, further comprising a second display routine for displaying an image icon indicating studies displayed in said image containers.

20. The set of instructions of claim 18, further comprising a third display routine for displaying a quick show window for displaying an information container from an image container.
